(19) United States
(12) Patent Application Publication
(10) Pub. No.: US 2019/0151379 A1
(43) Pub. Date: May 23, 2019

(54) ENZYME COMPOSITION AND USE THEREOF IN THE PREVENTION AND/OR TREATMENT OF GALECTIN-3 DEPENDENT DISORDERS

(71) Applicant: Pieter Muntendam, Boxford, MA (US)

(72) Inventor: Pieter Muntendam, Boxford, MA (US)

(21) Appl. No.: 16/197,000

(22) Filed: Nov. 20, 2018

Related U.S. Application Data
(60) Provisional application No. 62/590,012, filed on Nov. 22, 2017.

(51) Int. Cl. A61K 35/741; A61K 9/00; A61P 35/00; A61P 9/00; A61P 11/00; A23L 33/135; A23L 29/231; A61K 31/732

(52) U.S. Cl. CPC A61K 35/741; A61K 9/0053; A61P 35/00; A61K 31/732; A61P 11/00; A23L 33/135; A23L 29/231; A61P 9/00

(57) ABSTRACT

Methods for the prevention or treatment of a galectin-3 mediated or dependent condition in a subject are provided. The methods include orally administering a first amount of a carbohydrate material to the subject and orally administering a second amount of a selected carbohydrate digesting enzyme to the subject. The methods further include enzymatically converting the carbohydrate material into one or more bioactive fragments and producing the one or more bioactive fragments in an amount sufficient to therapeutically inhibit galectin-3 in the subject.

### RELATED APPLICATION

This application claims the benefit of priority to U.S. Provisional Application No. 62/590,012, filed on Nov. 22, 2017. The entire teachings of the above application are incorporated herein by reference.

### BACKGROUND OF THE INVENTION

Galectin-3 is a protein belonging to a specific sub-family of carbohydrate binding proteins (lectins) that recognize β-galactosides. Galectins possess a carbohydrate recognition domain (CRD). The CRDs of various galectins differ in amino acid sequence outside of the conserved residues and this mediates specificity to different glycan ligands between galectins. Galectin-3 has both intracellular functions and extracellular functions and is actively secreted via a non-canonical pathway into the extracellular space and into the circulation. Binding of carbohydrates to the CRD results in modulation of galectin-3 activity in-vitro and in-vivo. Carbohydrate binding to the CRD and the resulting inhibition of galectin-3 is recognized as a potential therapeutic modality.

Galectin-3 has multiple biological functions. Galectin-3 has been identified as a mediator of fibrosis. Galectin-3-mediated fibrosis has been found to be an important underlying cause of a broad range of disease manifestations affecting the heart, lungs, kidney, vascular and liver system. These conditions include serious chronic conditions such as heart failure, chronic kidney disease and pulmonary fibrosis. Galectin-3 has also been found to play a role in diseases affecting the brain, eye, skin, joints and other organs. Galectin-3 has also been found to play a role in cancer. Galectin-3 is considered an important potential therapeutic target. There are no therapeutic agents currently approved for prevention or treatment of galectin-3 mediated disorders.

Thus, there is an unmet need for the prevention or treatment of galectin-3 mediated disorders.

### SUMMARY OF THE INVENTION

In one aspect, the present disclosure relates to a method for inhibiting galectin-3 and/or for the treatment of galectin-3 mediated conditions in a subject in need thereof. The method includes orally administering to the subject an enzyme preparation containing carbohydrate digesting enzymes and carbohydrate-containing material. Particularly, the method includes orally administering a first amount of the carbohydrate material and a second amount of the selected carbohydrate digesting enzyme. The enzymes in the enzyme preparation are specifically selected for enzymatic conversion of the carbohydrate material into one or more bioactive fragments that can be absorbed from the gut and inhibit galectin-3. Both the enzymes preparation and the carbohydrate material are selected in combination to promote the formation of the one or more bioactive fragments. The method further includes producing the one or more bioactive fragments in an amount sufficient to therapeutically inhibit galectin-3 in the subject. In an embodiment, the subject may suffer from a galectin-3 associated disorder. The disorder may include heart disease, kidney disease, lung disease, liver disease, vascular disease or cancer. In an embodiment, the method includes co-administering the first amount of the carbohydrate material and the second amount of the selected carbohydrate digesting enzyme to the subject. In some embodiments, the method includes co-mingling the first amount of the carbohydrate material and the second amount of the selected carbohydrate digesting enzyme before administration to the subject.

The carbohydrate material, for example, pectins, pectic fragments or analogues can be ingested as a fruit, a vegetable, a nutritional product containing such materials, or by means of an oral dosage form of a pectin, pectin combination, pectic fragments or analogues. In various embodiments, the carbohydrate material may include one or more natural pectins, modified pectins, pectic fragments, pectic fragment derivatives, pectic fragment analogues, natural carbohydrate macromolecules, man-made carbohydrate macromolecules, edible fruits, vegetables, or combinations thereof.

Without wishing to be bound to any particular theory, it is believed that the enzymatic breakdown of the carbohydrate material will facilitate and improve absorption of the one or more bioactive fragments of the carbohydrate material and, hence, improve the therapeutic effect resulting from administration of the carbohydrate material. Enzymatic breakdown of the carbohydrate material in the gut can occur in the proximal part of the alimentary tract including the small intestine to increase the proportion of carbohydrate materials that are converted into bioactive molecules and their absorption. This would result in a shift from inefficient fermentation-based breakdown in the colon to a targeted enzymatic breakdown in the proximal part of the alimentary tract. This would enable increase in the percentage of the ingested carbohydrate material that would be converted into the desired bioactive moiety and, in certain cases, further enhanced through improved absorption taking place in both the small intestine and the colon.

Additionally, the enzymatic breakdown (also referred to herein as conversion or enzymatic conversion) may reduce the fermentation of the pectin or pectic material and hence reduce the incidence or severity of fermentation related bloating, flatulence, discomfort, and pain.

The improved therapeutic benefit per quantity of ingested carbohydrate material (therapeutic yield) as a result of targeted enzymatic breakdown and improved bioavailability would enable use of oral dosage forms currently not practical or feasible.

Accordingly, the present disclosure can provide methods for improving the efficacy and tolerance of treatment with ingested specific carbohydrate material such as a pectin by administering an enzymatic preparation in conjunction with the pectin administration. In various embodiments, the methods can include preventing or treating a galectin-3 mediated disorder such as heart failure, or kidney disease or alleviating one or more symptoms thereof in a patient. Such methods can include oral administration a therapeutically effective amount of a carbohydrate material and a specific enzyme preparation.

In some embodiments of the methods, the enzyme preparation includes a single enzyme, such as a pectin lyase. In certain embodiments, the enzyme preparation component includes multiple enzymes belonging to a certain pectinase class, such as multiple different pectin hydrolases. In yet another embodiment, the enzyme preparation may include one or more enzymes for each of multiple pectinase classes, such as a combination of two pectin lyases, and one pectin hydrolase. In one embodiment, the carbohydrate digesting enzyme may be derived from natural sources including one or more nonpathogenic bacteria, nonpathogenic fungi, or combinations thereof, or man-made sources including altered nonpathogenic bacteria, altered nonpathogenic fungi, or combinations thereof. In another embodiment, the carbohydrate digesting enzyme may be derived from natural sources including multiple species of nonpathogenic bacteria, nonpathogenic fungi, or combinations thereof. In yet another embodiment, the carbohydrate digesting enzyme may be derived from natural sources including at least one species of natural nonpathogenic bacteria, natural nonpathogenic fungi, or combinations thereof. In some embodiments, the carbohydrate digesting enzyme may be derived from man-made sources including at least one species of altered nonpathogenic bacteria, altered nonpathogenic fungi, or combinations thereof. The altered nonpathogenic bacteria or altered nonpathogenic fungi may be produced using bioengineering. In one embodiment, the carbohydrate digesting enzyme may be derived from sources including at least one species of nonpathogenic bacteria and at least one species of nonpathogenic fungi.

In various embodiments of the methods, the carbohydrate material component includes a natural plant product with a high content of such beneficial carbohydrate, a nutritional product containing a sufficient quantity of such material, a natural pectin, a modified pectin, a pectic fragment or a pectic derivative. In certain embodiments, the carbohydrate material includes man-made carbohydrate material. In certain embodiments, the carbohydrate material includes a combination of multiple different natural or man-made components identified and selected for its galectin-3 inhibition after ingestion and breakdown and absorption.

In another aspect, the present invention relates to a method of inhibiting galectin-3 in a subject suffering from a galectin-3 associated disorder. The disorder may include heart disease, kidney disease, lung disease, liver disease, vascular disease or cancer. The method includes co-mingling a first amount of a carbohydrate material and a second amount of a selected carbohydrate digesting enzyme to form a co-mingled material, and orally administering the co-mingled material to the subject. The method further includes enzymatically converting the co-mingled material into one or more bioactive fragments and producing the one or more bioactive fragments in an amount sufficient to therapeutically inhibit galectin-3 in the subject. In various embodiments, the carbohydrate material may include one or more natural pectins, modified pectins, pectic fragments, pectic fragment derivatives, pectic fragment analogues, natural carbohydrate macromolecules, man-made carbohydrate macromolecules, edible fruits, vegetables, or combinations thereof, and the selected carbohydrate digesting enzyme may be derived from sources including one or more nonpathogenic bacteria, nonpathogenic fungi, or combinations thereof.

In yet another aspect, the present disclosure includes a therapeutic combination, for example, a therapeutic preparation, including a pectinase and a modified pectin.

In yet another aspect, the present disclosure relates to a kit, where the kit includes a therapeutic combination that includes a first amount of a carbohydrate material and a second amount of a pectinase containing enzyme preparation or a carbohydrate digesting enzyme and instructions for use of the therapeutic combination having the first amount and the second amount.

The foregoing as well as other features and advantages of the present disclosure will be more fully understood from the following description, examples, and claims.

### DETAILED DESCRIPTION OF THE INVENTION

The present disclosure relates to a method for preventing or treating galectin-3 dependent disorders in a subject. The subject may refer to a patient, such as a human, who suffers from a galectin-3 associated disorders. The disorders may include heart disease, kidney disease, lung disease, liver disease, vascular disease or cancer. Specifically, the disclosure relates to the method of inhibiting galectin-3 in the subject by administering a composition of enzyme. The composition may include a carbohydrate material and a carbohydrate digesting enzyme. Particularly, the present disclosure relates to the oral administration of the carbohydrate material, for example, modified squash pectin, by combining the carbohydrate material with the enzyme preparation containing a pectin hydrolase and a pectin lyase.

In an embodiment, the method includes orally administering a first amount of the carbohydrate material to the subject and orally administering a second amount of the selected carbohydrate digesting enzyme to the subject. The enzyme preparation produces specific enzymes that enzymatically convert the carbohydrate material into one or more bioactive fragments. The method further includes producing the one or more bioactive fragments in an amount sufficient to therapeutically inhibit the galectin-3 in the subject. In another embodiment, the method includes co-administering the first amount of the carbohydrate material and the second amount of the carbohydrate digesting enzyme to the subject. In some embodiments, the method includes co-mingling the first amount of the carbohydrate material and the second amount of the carbohydrate digesting enzyme before administration to the subject.

Throughout the application, where carbohydrate materials are referenced, it is contemplated that compositions of the present disclosure includes specific natural or man-made carbohydrate macromolecules or combination of carbohydrate macromolecules that are specifically selected because breakdown yields bioactive fragments which can be absorbed and inhibit galectin-3 in mammals. In some embodiments, the carbohydrate material may include one or more natural pectins, modified pectins, pectic fragments, pectic fragment derivatives, pectic fragment analogues, natural carbohydrate macromolecules, man-made carbohydrate macromolecules, edible fruits, vegetables, or combinations thereof.

As used herein, “first amount” refers to an amount of the carbohydrate material that is sufficient to generate a therapeutically effective amount of the bioactive fragments for the treatment of galectin-3 associated disorders.

As used herein, “second amount” refers to an amount of the selected carbohydrate digesting enzyme that is sufficient to generate a therapeutically effective amount of the bioactive fragments for the treatment of galectin-3 associated disorders.

In some embodiments, the first amount of the carbohydrate material and the second amount of the selected carbohydrate digesting enzyme may be equal. In certain embodiments, the first amount of the carbohydrate material may be less than the second amount of the selected carbohydrate digesting enzyme. In various embodiments, the first amount of the carbohydrate material may be more than the second amount of the selected carbohydrate digesting enzyme.

Throughout the application, where enzyme preparations are referenced, it is contemplated that compositions of the present disclosure includes specific natural or man-made enzymes that are specifically selected because they cause enzymatic breakdown of the carbohydrate material in targeted bioactive fragments which can be absorbed and inhibit galectin-3.

Throughout the application, where galectin-3 mediated conditions or health and therapeutic benefits are referenced, it is contemplated that use of the products of the present disclosure results in the inhibition of galectin-3 mediated disease processes. In case of an established condition in a patient diagnosed with same, such as galectin-3 mediated heart failure, treatment with the products of the present disclosure resulting in inhibition of the galectin-3 mediated disease process which may result in halting the progression and improving the prognosis. In certain cases, use of the products of the present disclosure may allow recovery of function to take place which may result in a reduction in signs and symptoms and an improvement in functional status. When used in individuals with subclinical disease who have objective signs of the presence of the galectin-3 mediated disease processes, such as based on a blood test or imaging procedure or combination thereof, but not yet the clinical manifestations and corresponding diagnosis, treatment with the products of the present disclosure may reduce or arrest the disease processes so that onset of the clinical manifestations are delayed or avoided altogether. The understanding of the role of galectin-3 in disease processes is rapidly evolving with over 500 scientific articles referencing galectin-3 over the past 24 months. To date, the role of galectin-3 has been established in conditions affecting the heart (heart failure and atrial fibrillation), the kidney (chronic kidney disease), the lung (lung fibrosis), the vascular system (vascular stiffening), and the liver (liver fibrosis) where studies identified galectin-3 as a mediator of important disease processes and inhibition of galectin-3 by means of a carbohydrate inhibitor, such as modified citrus pectin (MCP), has been found to provide beneficial effects. Additionally, galectin-3 has been found to play a role in certain disease processes in cancer of various organs, in the brain, in the eye and the skin. Research may identify additional uses of the present disclosure in galectin-3 mediated conditions affecting these or other organs.

Throughout the application, where compositions are described as having, including, or comprising specific components, or where processes are described as having, including, or comprising specific process steps, it is contemplated that compositions of the present disclosure also includes the recited components, and that the processes of the present disclosure also includes the recited process steps.

In the application, where an element or component is said to be included in and/or selected from a list of recited elements or components, it should be understood that the element or component can be any one of the recited elements or components, or the element or component can be selected from a group including two or more of the recited elements or components. Further, it should be understood that elements and/or features of a composition, an apparatus, or a method described herein can be combined in a variety of ways without departing from the spirit and scope of the present disclosure, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise.

It should be understood that the order of steps or order for performing certain actions is immaterial so long as the present disclosure remains operable. Moreover, two or more steps or actions may be conducted simultaneously.

As used herein, “patient” refers to a mammal, such as a human

As used herein, a “compound” refers to the compound itself and its pharmaceutically acceptable salts, hydrates and esters, unless otherwise understood from the context of the description or expressly limited to one particular form of the compound, i.e., the compound itself, or a pharmaceutically acceptable salt, hydrate or ester thereof.

As used herein, “oral administration” refers to oral, preoral, buccal, sublabial, sublingual, and intraoral administration. The administration can be in the form of tablets, capsules, drops, liquid, syrups, powder, granules, softgel, or lozenges.

As used herein, “co-mingling” refers to combining or mixing or blending two or more components substantially completely, using either physical or chemical methods, such that a resulting material contains a substantially homogeneous combination or mixture or blend of the two or more components.

As used herein, an “enzyme preparation” refers to preparation containing one or more carbohydrate digesting enzymes, when administered in adequate amounts, breaks down complex carbohydrates into fragments including the targeted bioactive fragments which confer a health benefit on the host. Enzymes are macromolecular biological catalysts which accelerate chemical reactions. The targeted action is a controlled breakdown of carbohydrate material into fragments in a manner that yields bioactive carbohydrate fragments in sufficient quantity to confer a health benefit. In one embodiment, the carbohydrate digesting enzyme may be derived from natural sources including one or more nonpathogenic bacteria, nonpathogenic fungi, or combinations thereof, or man-made sources including altered nonpathogenic bacteria, altered nonpathogenic fungi, or combinations thereof. In another embodiment, the carbohydrate digesting enzyme may be derived from natural sources including multiple species of nonpathogenic bacteria, nonpathogenic fungi, or combinations thereof. In yet another embodiment, the carbohydrate digesting enzyme may be derived from natural sources including at least one species of natural nonpathogenic bacteria, natural nonpathogenic fungi, or combinations thereof. In some embodiments, the carbohydrate digesting enzyme may be derived from man-made sources including at least one species of altered nonpathogenic bacteria, altered nonpathogenic fungi, or combinations thereof. The altered nonpathogenic bacteria or altered nonpathogenic fungi may be produced using bioengineering. In one embodiment, the carbohydrate digesting enzyme may be derived from sources including at least one species of nonpathogenic bacteria and at least one species of nonpathogenic fungi.

As used herein, “nonpathogenic bacteria” refers to any such bacteria that are nonpathogenic in nature and are to produce pectinases. Such nonpathogenic bacteria may include strains of Bacillus, Erwinia, Pseudomonas and any other nonpathogenic pectinase producing bacterial strains.

As used herein, “nonpathogenic fungi” refers to any such fungi that are nonpathogenic in nature and are to produce pectinases strains of Aspergillus, Kluyveromyces, Rhizopus, Trichoderma and other nonpathogenic pectinase producing fungal strains.

As used herein, a “carbohydrate material” refers to a compound which after breakdown yields one or more fragments that are enterally absorbed and bind to the galectin-3 CRD resulting in inhibition of galectin-3 activity. In one embodiment, the carbohydrate material may be a natural pectin. In another embodiment, the carbohydrate material may be a modified pectin. In yet another embodiment, the carbohydrate material may be a pectic fragment. In yet another embodiment, the carbohydrate material may be a pectic fragment derivative or analogues. In yet another embodiment, the carbohydrate material may be a man-made carbohydrate macromolecule. A carbohydrate material can also include a fruit or vegetable, a part of a fruit or vegetable, a nutritional product which includes sufficient quantities of the carbohydrate material, a powder or other ingestible product that can be consumed in conjunction with other foods, a drink, a therapeutic preparation including the natural pectin or the modified pectin, a therapeutic preparation including an artificial or man-made carbohydrate macromolecule, pectic derivative or pectic analogue, a therapeutic preparation including the pectic fragment, or a therapeutic preparation including product including a not naturally occurring carbohydrate or analogue thereof.

As used herein, “enzymatic conversion” refers to conversion of the carbohydrate material into the one or more bioactive fragments by enzymes. The enzymes may include pectinases, hydrolases, esterases, lyases, transferases, amylases, cellulases, xylanases, invertases, inulinases, glycosidases, and epimerases.

As used herein, “bioactive fragments” refers to any such fragments generated after the breakdown of carbohydrates, which may include bioactive polysaccharides, monosaccharides, disaccharides, oligosaccharides and any such fragment resulting from the breakdown processes that can produce a desired therapeutic effect.

As used herein, “man-made carbohydrate macromolecule” refers to all such carbohydrate macromolecules that are synthesized by various physical, chemical, enzymatic, and biochemical processes. The processes may include addition, radical reaction, condensation, wittig reaction, and other such processes that modify the structure of naturally occurring carbohydrates and result in a new macromolecule.

Pectins are complex carbohydrates that are contained in the primary cell walls of terrestrial plants. Pectins are among the most heterogenous compounds known to man Certain pectins and pectic fragments are known to bind to galectin-3 and inhibit galectin-3 activity.

Pectins can be broken down by enzymes known as pectinases. Numerous pectinases have been identified and characterized. Pectinases differ as to the nature of the breakdown and the resulting pectic fragments. Naturally occurring pectinases are found in certain fungi or bacteria. Pectinases are commonly used in the production of nutritional products, such as fruit juice or wine. Although the term pectinases are used herein, these enzymes break down certain carbohydrate materials regardless of their origin, including man-made carbohydrate material.

Pectins and pectin-like carbohydrates are too large for absorption in the gut. Mammals do not produce pectinases and are hence dependent upon the gut flora for break-down of ingested pectins. The gut flora is highly variable within an individual, for example in response to changes in diet or occurrence of disease, and between individuals. Breakdown of pectins in the gut is generally confined to the colon where pectins are broken-down through fermentation. Fermentation is a non-specific breakdown by microbes. Colonic fermentation yields both gases (including hydrogen, methane, and carbon dioxide) and short-chain fatty acids (SCFAs; including acetic, propionic, and butyric acids). Fermentation of pectins and the resulting gasses may cause bloating, discomfort, abdominal cramps, flatulence and pain.

In order to identify suitable carbohydrate material for the present disclosure experiments and trials can be conducted which include using laboratory methods to breakdown the pectin, such methods may include the use of physical methods (e.g. heating, centrifuge, ultrasound), use of chemical methods (e.g. use of acids, alkaline materials or other chemicals, or use of enzymes, or a combination of such methods to yield a library of fragments. These fragments can be further characterized by suitable laboratory methods. The ability of fragments to inhibit galectin-3 can be investigated through use of one or more techniques, such as binding assays or spectroscopy. If the fragment displays the binding characteristics suggestive of inhibition of activity, in-vitro or in-vivo experiments maybe conducted to test and characterize the degree of galectin-3 inhibition by the fragment. If the observed inhibition suggests the possible use in the combination product of the present disclosure, the oral absorption of the fragments may be investigated by oral administration of a known quantity to an animal in a model suitable for such investigation, or by investigating this in experiments that may be used as a surrogate for traversing the intestinal wall resulting in absorption. If such experiment suggests oral absorption of the fragment sufficient to expect a therapeutic benefit, experiments maybe conducted on the use of pectinases to breakdown the carbohydrate material. Pectinases used in such experimentation maybe manufactured in the laboratory or obtained from a commercial source. Once a pectinase or combination of pectinases has been identified which breaks down the carbohydrate material in a way that among others yields the desired bioactive moiety or moieties, studies may be initiated that mimic how oral ingestion of these enzymes affects intestinal breakdown of the carbohydrate material. The gastro-intestinal milieu may substantially enhance or diminish the effect of the enzymes on the carbohydrate material, for example through a different pH than was used in the experimental conditions. Enzymes suitable for the present disclosure must be toxic. Additionally, enzymes must be suitable for human use which requires ability to be manufactured at consistent quality and at commercial scale, be stable in a suitable presentation, preferably at room temperature without special preservatives, and be able to function in the gut in a manner that facilitates breakdown of the carbohydrate material in the gut of humans under typical circumstances. Once enzymes have been identified that based on these and other criteria are deemed suitable for the present disclosure, these can be investigated through laboratory and in-vivo experiments. Enzymes for use in the present disclosure can also be developed through use of bioengineering techniques, such as gene editing. The laboratory experiment may investigate the composition of present disclosure in a laboratory experiment that mimics what may happen in parts of the alimentary track to investigate if the combination product yields the desired fragments. The laboratory experiment may compare the yield of the desired bioactive galectin-3 binding moieties after ingestion of the selected carbohydrate material with and without the administration of the enzyme preparation component of the teaching. The yield of desired galectin-3 inhibiting bioactive moieties would be increased if the developed enzyme preparation\component is active in accordance with the current disclosure. Development steps and experiments may need to be repeated to achieve at the desired characteristics of the combination product. Additional methods of investigating may include administration of the combination product to a mammal in a suitable experiment and measuring the presence of the bioactive moieties in bodily fluids of the animal. Such experiments may compare the levels of the moiety or moieties in the bodily fluids after ingesting of the combination product in comparison to ingestion of the carbohydrate material in absence of the enzyme preparation. Elevated levels of the moiety or extent of exposure (area under the curve) indicate that the combination is effective in accordance with the current disclosure.

As used herein, “natural pectin” refers to complex carbohydrates that are contained in the primary cell walls of terrestrial plants. Pectins are among the most heterogenous compounds known to man Certain pectins and pectic fragments are known to bind to galectin-3 and inhibit galectin-3 activity.

As used herein, “derived” refers to methods of converting a starting material to a corresponding derivative or product as desired, using chemical, enzymatic, biotechnological, biochemical, bioconversion, physical or any combinations of such methods. Such methods may include, but are not limited to, use of microbial or chemical fermentation, use of hydrolases, isomerases, transferases, esterases, dehydrogenases, and oxidases, or use of chemical processes such as, oxidation, esterification, etherification, acid hydrolysis, cross-linking, and cationization, or employing physical processes such as, annealing, extrusion, heat-moisture treatment and any combinations of such processes.

As used herein, “modified” refers to a derivative formed by various methods or processes including, but not limited to, substitutions such as alkylation, amidation, quaternization, thiolation, sulfation, and oxidation, chain elongations such as cross-linking and grafting, and depolymerization by chemical, physical, or biological including enzymatic means. The methods and processes can be employed either alone or in any combination without any specific order.

As used herein, “modified pectin” refers to pectin modified by substitution, chain elongation, depolymerization, saponification, enzymes, salts of weak acids, bases, mineral acids, concentrated ammonium systems and primary aliphatic amines or by any other chemical, enzymatic, biochemical and physical methods.

As used herein, “pectic fragment” refers to any part of the pectic macromolecule derived after subjecting pectic polysaccharide to a breakdown process. The resulting fragments may include galacturonans such as unsubstituted homogalacturonan (HG), rhamnogalacturonan II (RG-II), xylogalacturonan (XGA), Arabinan, Arabinogalactan I, Arabinogalactan II, α-L-arabinofuranose, α-D-galactopyranose (rhamnogalacturonan I), other oligosaccharides, structural elements and side chains.

As used herein, “pectic fragment derivative” refers to any such derivatives that result from subjecting any pectic fragments representing all three major types of pectic polysaccharide, homogalacturonan (HG), rhamnogalacturonan-I (RG-I) and rhamnogalacturonan-II (RG-II) to modification processes. The resulting pectic fragment derivatives may include fragments of homogalacturonan such as oligomers, oligogalacturonic acids, di-galacturonic acids, tri-galacturonic acids, and polygalacturonic acids, fragments of Rhamnogalacturonan-I such as disaccharide [→4)-α-d-Gal p A-(1→2)-α-1-Rha p], oligomers of 1-5 disaccharide repeat units, Rhamnogalacturonan-II side chain A fragments, and any such fragments resulting from the breakdown of the major pectic polysaccharides.

As used herein, “therapeutic combination” refers to a combination of an enzyme preparation and a carbohydrate material. Typically, each such component in the therapeutic combinations of the present disclosure can be present in a pharmaceutical composition comprising that ingredient and a pharmaceutically acceptable carrier. Additionally, the carbohydrate material can be in the form of a nutritional product with or without the enzyme preparation component mixed in. The compounds in a therapeutic combination of the present disclosure can be administered simultaneously, together or separately, or separately at different times, as part of a regimen.

The present disclosure also provides pharmaceutical compositions that include at least one compound described herein or a therapeutic combination, and one or more pharmaceutically acceptable carriers, excipients, or diluents. Examples of such carriers are well known to those skilled in the art and can be prepared in accordance with acceptable pharmaceutical procedures, such as, for example, those described in Remington: The Science and Practice of Pharmacy, 20th edition, ed. Alfonso R. Gennaro, Lippincott Williams & Wilkins, Baltimore, Md. (2000), the entire disclosure of which is incorporated by reference herein for all purposes.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds and therapeutic combinations of the present disclosure can be useful for preventing or treating a pathological condition or disorder in a patient, for example, a human. As used herein, “preventing or treating” refers to partially or completely alleviating and/or ameliorating the condition and/or symptoms thereof, and/or preventing its occurrence or halting its progression. The present disclosure accordingly includes a method of providing to a patient a combination product that includes a compound or therapeutic combination of the present disclosure in combination or association with a pharmaceutically acceptable carrier. Compounds and therapeutic combinations of the present disclosure can be administered alone or in combination with other therapeutically effective compounds or therapies for the treatment of a pathological condition or disorder.

As used herein, “therapeutically effective” refers to a substance or an amount that elicits a desirable biological activity or effect through inhibition of galectin-3 and reducing or arresting galectin-3 mediated disease processes.

As used herein, “therapeutically effective amount” refers to an amount that may elicit a desired level of the desired biological or medical response in a tissue, system, animal, or human by the researcher, veterinarian, medical doctor, or other clinician. As used herein, a therapeutically effective amount also refers to the quantity required to achieve a desired therapeutic or prophylactic effect.

When administered for the treatment or inhibition of a particular galectin-3 mediated disease state or disorder, it is understood that an effective dosage can vary depending upon many factors such as the particular compound or therapeutic combination utilized, the mode of administration, and severity of the condition being treated, as well as the various physical factors related to the individual being treated. In therapeutic applications, a compound or therapeutic combination of the present disclosure can be provided to a patient already suffering from a disease, for example, heart failure, in an amount sufficient to at least partially ameliorate the symptoms of the disease and its complications and halt or slow down the disease progression. If administered to a human suffering from the condition prior to clinical manifestation, the administration of a therapeutic combination may prevent the first clinical manifestation or delay its onset.

The compounds and therapeutic combinations described herein are administered orally in solid, liquid or powder form where each of the components is represented in the same physical form (alone or in combination) or in a different one, such as where the enzyme preparation is presented in powder form in a capsule and the carbohydrate material in the form of a liquid to be consumed following the ingestion of the enzyme preparation.

The compounds and therapeutic combinations described herein are administered orally in solid, liquid or powder form where the combination may be combined with other ingredients outside the present disclosure. Such ingredients may be included for a variety of reasons including to further enhance the breakdown of the carbohydrate material in the combination into the bioactive fragments.

In another embodiment, the present disclosure relates to the method of inhibiting galectin-3 in the subject suffering from the galectin-3 associated disorder. The disorder may include heart disease, kidney disease, lung disease, liver disease, vascular disease or cancer. The method includes co-mingling the first amount of the carbohydrate material and the second amount of the selected carbohydrate digesting enzyme to form a co-mingled material. The carbohydrate material may include one or more natural pectins, modified pectins, pectic fragments, pectic fragment derivatives, pectic fragment analogues, natural carbohydrate macromolecules, man-made carbohydrate macromolecules, edible fruits, vegetables, or combinations thereof. The selected carbohydrate digesting enzyme may be derived from sources including one or more nonpathogenic bacteria, nonpathogenic fungi, or combinations thereof. The method further includes orally administering the co-mingled material to the subject and enzymatically converting the co-mingled material into the one or more bioactive fragments. The method further includes producing the one or more bioactive fragments in an amount sufficient to therapeutically inhibit the galectin-3 in the subject.

In yet another embodiment, the present teaching relates to a kit having the first amount of the carbohydrate material and the second amount of the carbohydrate digesting enzyme. The kit may also include instructions for use of the first amount of the carbohydrate material and the second amount of the carbohydrate digesting enzyme.

The following examples are provided to illustrate further and to facilitate the understanding of the present disclosure and are not in any way intended to limit the invention.

### EXAMPLE 1: PATIENT WITH EARLY ONSET HEART FAILURE

A patient is his late 60's with a confirmed diagnosis of heart failure two years after a myocardial infarction was found to have elevated galectin-3 levels (29.6 ng/mL) and elevated collagen turn-over markers, indicating that galectin-3 mediated disease process may have caused or contributed to the onset and progression of the disease. The patient was recently admitted to a hospital for fluid overload. The patient is on guideline recommended heart failure background therapy. The patient is prescribed the galectin-3 inhibiting combination including an enzyme preparation containing three different enzymes belonging to two classes of pectinases and a modified pectin in this case squash pectin. The combination was designed to breakdown the squash pectin in known bioactive moieties that are absorbed in the gut to enable the therapeutic benefit. The two products are combined in a therapeutic preparation including a capsule. The patient is prescribed chronic therapy including two capsules twice a day.

### EXAMPLE 2: PATIENT WITH CHRONIC KIDNEY DISEASE

A patient in his mid 40's with a history of hypertension was recently diagnosed with early-onset chronic kidney disease indicated by reduced and gradually worsening eGFR and elevated levels of galectin-3 (29.4 ng/mL). The patient is prescribed an oral regimen. The patient is prescribed the combination including an enzyme preparation and a modified pectin in this case modified apple pectin. The enzyme preparation is taken as a capsule to be taken once a day on an empty stomach. The modified apple pectin is in a powder presentation to be taken with a meal within one hour of the enzyme preparation, such as over cereal for breakfast. The patient is prescribed chronic therapy including this combination.

### EXAMPLE 3: PATIENT WITH ELEVATED GALECTIN-3

A patient in his early fifties a family history of heart disease including heart failure was found to have increasing levels of galectin-3. In recent years plasma levels of galectin-3 increased from around 10 ng/mL to 19.6 ng/mL. The patient is prescribed an enzyme preparation including three pectinases with different pectinolytic activities selected to break down dietary pectins in bioactive fragments that inhibit galectin-3. The patient was instructed to take the enzyme preparation three times daily. The patient is prescribed a diet rich in certain fruit and vegetables that are known to break down into galectin-3 inhibiting bioactive fragments when enzymatically broken down by the pectinases in the prescribed combination.

The present disclosure encompasses embodiments in other specific forms without departing from the spirit or essential characteristics thereof. The foregoing embodiments are therefore to be considered in all respects illustrative rather than limiting on the present disclosure described herein. Scope of the present invention is thus indicated by the appended claims rather than by the foregoing description, and all changes that come within the meaning and range of equivalency of the claims are intended to be embraced therein.

What is claimed is:

1. A method of inhibiting galectin-3 in a subject in need thereof comprising:
orally administering to the subject, a first amount of a carbohydrate material;
orally administering to the subject, a second amount of a selected carbohydrate digesting enzyme;
enzymatically converting the carbohydrate material into one or more bioactive fragments; and
producing the one or more bioactive fragments in an amount sufficient to therapeutically inhibit galectin-3 in the subject.

2. The method of claim 1 further comprising, co-administering the first amount of the carbohydrate material and the second amount of the selected carbohydrate digesting enzyme.

3. The method of claim 2 further comprising, co-mingling the first amount of the carbohydrate material and the second amount of the selected carbohydrate digesting enzyme before administration.

4. The method of claim 1, wherein the subject is suffering from a galectin-3 associated disorder.

5. The method of claim 4, wherein the disorder comprises heart disease, kidney disease, lung disease, liver disease, vascular disease or cancer.

6. The method of claim 1, wherein the carbohydrate material comprises one or more natural pectins, modified pectins, pectic fragments, pectic fragment derivatives, pectic fragment analogues, natural carbohydrate macromolecules, man-made carbohydrate macromolecules, edible fruits, vegetables, or combinations thereof.

7. The method of claim 1, wherein the carbohydrate material is a natural pectin.

8. The method of claim 1, wherein the carbohydrate material is a modified pectin.

9. The method of claim 1, wherein the carbohydrate material is a pectic fragment.

10. The method of claim 1, wherein the carbohydrate material is a pectic fragment derivative or analogue.

11. The method of claim 1, wherein the carbohydrate material is a man-made carbohydrate macromolecule.

12. The method of claim 1, wherein the carbohydrate digesting enzyme is derived from:
natural sources comprising one or more nonpathogenic bacteria, nonpathogenic fungi, or combinations thereof; or
man-made sources comprising altered nonpathogenic bacteria, altered nonpathogenic fungi, or combinations thereof.

13. The method of claim 12, wherein the carbohydrate digesting enzyme is derived from natural sources comprising multiple species of nonpathogenic bacteria, nonpathogenic fungi, or combinations thereof.

14. The method of claim 12, wherein the carbohydrate digesting enzyme is derived from natural sources comprising at least one species of natural nonpathogenic bacteria, natural nonpathogenic fungi, or combinations thereof.

15. The method of claim 12, wherein the carbohydrate digesting enzyme is derived from man-made sources comprising at least one species of altered nonpathogenic bacteria, altered nonpathogenic fungi, or combinations thereof.

16. The method of claim 15 further comprising, producing the altered nonpathogenic bacteria or altered nonpathogenic fungi using bioengineering.

17. The method of claim 12, wherein the carbohydrate digesting enzyme is derived from sources comprising at least one species of nonpathogenic bacteria and at least one species of nonpathogenic fungi.

18. A method of inhibiting galectin-3 in a subject suffering from a galectin-3 associated disorder, the method comprising:
co-mingling a first amount of a carbohydrate material and a second amount of a selected carbohydrate digesting enzyme to form a co-mingled material;
orally administering to the subject, the co-mingled material;
enzymatically converting the co-mingled material into one or more bioactive fragments; and
producing the one or more bioactive fragments in an amount sufficient to therapeutically inhibit galectin-3 in the subject.

19. The method of claim 18, wherein the disorder is selected from heart disease, kidney disease, lung disease, liver disease, vascular disease and cancer; wherein the carbohydrate material comprises one or more natural pectins, modified pectins, pectic fragments, pectic fragment derivatives, pectic fragment analogs, natural carbohydrate macromolecules, man-made carbohydrate macromolecules, edible fruits, vegetables, or combinations thereof; and wherein the carbohydrate digesting enzyme is derived from sources comprising one or more nonpathogenic bacteria, nonpathogenic fungi, or combinations thereof.

20. A kit comprising a first amount of a carbohydrate material and a second amount of a selected carbohydrate digesting enzyme and instructions for use of the first and second amounts.
